(19) United States
(12) Patent Application Publication
(10) Pub. No.: US 2010/0316573 A1
(43) Pub. Date: Dec. 16, 2010

(54) Organic Compounds

(76) Inventors: Larry Alexander Gaither, Cambridge, MA (US); Vadim Iourgenko, Cambridge, MA (US); Mark Aron Labow, Cambridge, MA (US); Dale Alan Porter, Cambridge, MA (US); Christopher Sean Straub, Cambridge, MA (US); Yao Yao, Cambridge, MA (US); Leigh Zawel, Cambridge, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/446,430

(22) PCT Filed: Oct. 17, 2007
(86) PCT No.: PCT/US07/22125

§ 371 Date: Apr. 20, 2009

Related U.S. Application Data
(60) Provisional application No. 60/862,155, filed on Oct. 19, 2006.
(60) Provisional application No. 60/862,161, filed on Oct. 19, 2006.

(51) Int. Cl. A61K 49/00; A61K 31/4439; C07D 401/02; A61P 9/00; A61P 29/00; A61P 35/00; A61P 37/00

(52) U.S. Cl. 424/9.2; 514/343; 546/278.4

(57) ABSTRACT

A method to predict which patients will respond to a IAP inhibiting compound comprising:
a) administering an IAP inhibitor compound to a patient, and
b) measuring TNF-α or IL-β levels.

### BACKGROUND OF THE INVENTION

Tumor necrosis factor alpha (TNF-α) is a cytokine that is released primarily by inflammation and mononuclear phagocytes in response to immunostimulators. TNF-α is capable of enhancing most cellular processes, such as differentiation, recruitment, proliferation, and proteolytic degradation. At low levels, TNF-α confers protection against infective agents, tumors, and tissue damage. However, TNF-α also has a role in many diseases. When administered to mammals such as humans, TNF-α causes or aggravates inflammation, fever, cardiovascular effects, hemorrhage, coagulation, and acute phase responses similar to those seen during acute infections and shock states. Enhanced or unregulated TNF-α production has been implicated in a number of diseases and medical conditions, for example, cancers, such as solid tumors and blood-born tumors; heart disease, such as congestive heart failure; and viral, genetic, inflammatory, allergic, and autoimmune diseases.

It has been found that in tumor cell lines which are highly sensitive (IC₅₀<500 nM) to compounds which inhibit the binding of the Smac protein to IAP (hereinafter “IAP Inhibitor compounds”) as single agents, anti-tumor activity results from the release of a block to a proapoptotic autocrine TNF-α signaling loop. The coordinate consequences of releasing this block are an increase in the production of TNFα and facilitation of TNFα-mediated apoptosis. Proliferative diseases within the scope of the present invention are those where TNFα signaling is constitutively on.

It is not known at this time how the IAP inhibitor compounds regulate the levels of TNF-α. However, since the cytokine IL-8 is produced in response to TNFα, cytokine levels (i.e., IL-8) in circulating blood may reflect therapeutic effect of IAP Inhibitor compounds and thus may be used as biomarkers.

The invention also relates to methods to predict the responsiveness of a patient with a TNF-α responsive disease to a IAP inhibitor compound. In particular, this invention relates to predicting a patient's response to an IAP inhibitor compound by measuring TNF-α levels, possibly pre- and post-treatment.

### SUMMARY OF THE INVENTION

The present invention, as described herein below overcomes deficiencies in the use of IAP inhibitor compounds by providing a method to determine which individual with a disease characterized by constitutive TNF-α signaling will respond to treatment with a IAP inhibitor compound.

In another embodiment, the present invention relates to the use of compounds that inhibit the binding of the Smac protein to IAPs (“AP inhibitor”) for the treatment of diseases characterized by constitutive TNF-α signaling, and to a method for the manufacture of a medicament for treating diseases characterized by constitutive TNF-α signaling, and to a method for the treatment of warm-blooded animals, including humans, wherein an IAP inhibitor is administered to a warm-blooded animal suffering diseases characterized by constitutive TNF-α signaling, especially proliferative diseases effected by cytokine production such as cancer, arthritis, sepsis, cancer associated cachexia, Crohn's disease and other inflammatory disorders.

### DESCRIPTION OF THE FIGURES

FIG. 1 shows (a) a correlation between sensitivity to Compound II and TNF mRNA levels within a panel of tumor cell lines. (b) that tumor cell lines which are sensitive to IAP inhibitor compounds are induced to increase TNF mRNA levels as part of their response.

FIG. 2 shows the increase of TNFα mRNA correlating to compounds II and III in SKOV-3 cells in a dose dependent manner.

### DETAILED DESCRIPTION OF THE INVENTION

One embodiment this invention provides a method to predict which patients will respond to a IAP inhibitor compound in patients having a disease characterized by constitutive TNF-α signaling comprising:

a) administering an IAP inhibitor compound to a patient, and

b) measuring TNF-α and/or IL-8 levels in said patient.

If the TNF-α levels in the patient increase upon administration of the IAP inhibitor compound, this is an indication that the compound is working.

In another embodiment, the present invention relates to the use of compounds that inhibit the binding of the Smac protein to IAPs (“IAP inhibitors”) to manufacture a medicament for the treatment of diseases characterized by constitutive TNF-α signaling.

The present invention also relates to a method to treat diseases characterized by constitutive TNF-α signaling by administering IAPs inhibitors in combination with TNF-α, Interferon-alpha or Interferon-gamma or other agents which modulate TNF-α signaling.

Examples of IAP inhibitors for use in the present invention include compounds of formula I:

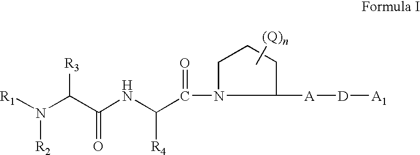

or pharmaceutically acceptable salts thereof, wherein

R₁ is H, C₁-C₄ alkyl, C₂-C₄ alkenyl, C₂-C₄ alkynyl or C₃-C₁₀ cycloalkyl, which R₁ may be unsubstituted or substituted;

R₂ is H, C₁-C₄ alkyl, C₂-C₄ alkenyl, C₂-C₄ alkynyl, C₃-C₁₀ cycloalkyl which R₂ may be unsubstituted or substituted;

R₃ is H, CF₃, C₂F₅, C₁-C₄ alkyl, C₂-C₄ alkenyl, C₂-C₄ alkynyl, CH₂-Z or R₂ and R₃ taken together with the nitrogen atom to which they are attached form a heterocyclic ring, which alkyl, alkenyl, alkynyl or het ring may be unsubstituted or substituted;

Z is H, OH, F, Cl, CH₃, CH₂Cl, CH₂F or CH₂OH;

R₄ is C₀₋₁₀ alkyl, C₃-C₁₀ cycloalkyl, wherein the C₀₋₁₀ alkyl, or cycloalkyl group is unsubstituted or substituted;

A is het, which may be substituted or unsubstituted;

D is C₁-C₇ alkylene or C₂-C₉ alkenylene, C(O), O, NR₇, S(O)ᵣ, C(O)—C₁-C₁₀ alkyl, O—C₁-C₁₀ alkyl, S(O)ᵣ—C₁-C₁₀ alkyl, C(O)C₀-C₁₀ arylalkyl C₀-C₁₀ arylalkyl, or S(O)r C₀-C₁₀ arylalkyl, which alkyl and aryl groups may be unsubstituted or substituted;

r is 0, 1, or 2;

A₁ is a substituted aryl or unsubstituted or substituted het which substituents on aryl and het are halo, lower alkoxy, NR₅R₆, CN, NO₂ or SR₅;

each Q is independently H, C₁-C₁₀ alkyl, C₁-C₁₀ alkoxy, aryl C₁-C₁₀ alkoxy, OH, O—C₁-C₁₀-alkyl, (CH₂)₀₋₆—C₃-C₇ cycloalkyl, aryl, aryl C₁-C₁₀ alkyl, O—(CH₂)₀₋₆ aryl, (CH₂)₁₋₆het, het, O—(CH₂)₁₋₆het, —OR₁₁, C(O)R₁₁, —C(O)N(R₁₁)(R₁₂), N(R₁₁)(R₁₂), SR₁₁, S(O)R₁₁, S(O)₂ R₁₁, S(O)₂—N(R₁₁)(R₁₂), or NR₁₁—S(O)₂—(R₁₂), wherein alkyl, cycloalkyl and aryl are unsubstituted or substituted;

n is 0, 1, 2 or 3, 4, 5, 6 or 7;

het is a 5-7 membered monocyclic heterocyclic ring containing 1-4 heteroring atoms selected from N, O and S or an 8-12 membered fused ring system that includes one 5-7 membered monocyclic heterocyclic ring containing 1, 2, or 3 heteroring atoms selected from N, O and S, which het is unsubstituted or substituted;

R₁₁ and R₁₂ are independently H, C₁-C₁₀ alkyl, (CH₂)₀₋₆—C₃-C₇cycloalkyl, (CH₂)₀₋₆—(CH)₀₋₁(aryl)₁₋₂, C(O)—C₁-C₁₀alkyl, —C(O)—(CH₂)₁₋₆—C₃-C₇cycloalkyl, —C(O)—O—(CH₂)₀₋₆-aryl, —C(O)—(CH₂)₀₋₆—O-fluorenyl, C(O)—NH—(CH₂)₀₋₆-aryl, C(O)—(CH₂)₀₋₆-aryl, C(O)—(CH₂)₁₋₆-het, —C(S)—C₁-C₁₀alkyl, —C(S)—(CH₂)₁₋₆—C₃-C₇cycloalkyl, —C(S)—O—(CH₂)₀₋₆-aryl, —C(S)—(CH₂)₀₋₆—O-fluorenyl, C(S)—NH—(CH₂)₀₋₆-aryl, —C(S)—(CH₂)₀₋₆-aryl or C(S)—(CH₂)₁₋₆-het, C(O)R₁₁, C(O)NR₁₁R₁₁R₁₂, C(O)OR₁₁, S(O)nR₁₁, S(O)mNR₁₁R₁₂, m=1 or 2, C(S)R₁₁, C(S)NR₁₁R₁₂, C(S)OR₁₁, wherein alkyl, cycloalkyl and aryl are unsubstituted or substituted; or R₁₁ and R₁₂ are a substituent that facilitates transport of the molecule across a cell membrane; or R₁₁ and R₁₂ together with the nitrogen atom form het;

wherein the alkyl substituents of R₁₁ and R₁₂ may be unsubstituted or substituted by one or more substituents selected from C₁-C₁₀alkyl, halogen, OH, O—C₁-C₆alkyl, CF₃ or NR₁₁R₁₂;
substituted cycloalkyl substituents of R₁₁ and R₁₂ are substituted by one or more substituents selected from a C₂-C₁₀ alkene; C₁-C₆alkyl; halogen; OH; O—C₁-C₆alkyl; S—C₁-C₆alkyl, CF₃; or NR₁₁R₁₂ and
substituted het or substituted aryl of R₁₁ and R₁₂ are substituted by one or more substituents selected from halogen, hydroxy, C₁-C₄ alkyl, C₁-C₄ alkoxy, nitro, CN O—C(O)—C₁-C₄alkyl and C(O)—O—C₁-C₄-alkyl;

R₅, R₆ and R₇ are independently hydrogen, lower alkyl, aryl, aryl lower alkyl, cycloalkyl; or cycloalkyl lower alkyl, and

wherein the substituents on R₁, R₂, R₃, R₄, Q, and A and A₁ groups are independently halo, hydroxy, lower alkyl, lower alkenyl, lower alkynyl, lower alkanoyl, lower alkoxy, aryl, aryl lower alkyl, amino, amino lower alkyl, diloweralkylamino, lower alkanoyl, amino lower alkoxy, nitro, cyano, cyano lower alkyl, carboxy, lower carbalkoxy, lower alkanoyl, aryloyl, lower arylalkanoyl, carbamoyl, N-mono- or N,N-dilower alkyl carbamoyl, lower alkyl carbamic acid ester, amidino, guanidine, ureido, mercapto, sulfo, lower alkylthio, sulfoamino, sulfonamide, benzosulfonamide, sulfonate, sulfanyl lower alkyl, aryl sulfonamide, halogen substituted aryl sulfonate, lower alkylsulfinyl, arylsulfinyl; aryl-lower alkylsulfinyl, lower alkylarylsulfinyl, lower alkylsulfonyl, arylsulfonyl, aryl-lower alkylsulfonyl, lower aryl alkyl lower alkylarylsulfonyl, halogen-lower alkylmercapto, halogen-lower alkylsulfonyl, phosphono (—P(═O)(OH)₂), hydroxy-lower alkoxy phosphoryl or di-lower alkoxyphosphoryl, (R₉)NC(O)—NR₁₀R₁₃, lower alkyl carbamic acid ester or carbamates or —NR₈R₁₄, wherein R₈ and R₁₄ can be the same or different and are independently H or lower alkyl, or R₈ and R₁₄ together with the N atom form a 3- to 8-membered heterocyclic ring containing a nitrogen heteroring atoms and may optionally contain one or two additional heteroring atoms selected from nitrogen, oxygen and sulfur, which heterocyclic ring may be unsubstituted or substituted with lower alkyl, halo, lower alkenyl, lower alkynyl, hydroxy, lower alkoxy, nitro, amino, lower alkyl, amino, diloweralkyl amino, cyano, carboxy, lower carbalkoxy, formyl, lower alkanoyl, oxo, carbarmoyl, N-lower or N,N-dilower alkyl carbamoyl, mercapto, or lower alkylthio, and

R₉, R₁₀, and R₁₃ are independently hydrogen, lower alkyl, halogen substituted lower alkyl, aryl, aryl lower alkyl, halogen substituted aryl, halogen substituted aryl lower alkyl. Compounds within the scope of formula (I) and the process for their manufacture are disclosed in U.S. 60/835,000, which is hereby incorporated into the present application by reference. The preferred compounds are selected from the group consisting of (S)—N—((S)-1-Cyclohexyl-2-{(S)-2-[4-(4-fluoro-benzoyl)-thiazol-2-yl]-pyrrolidin-1-yl}-2-oxo-ethyl)-2-methylamino-propionamide (Compound II); (S)—N—RS)-Cyclohexyl-(ethyl-{(S)-1-[5-(4-fluoro-benzoyl)-pyridin-3-yl]-propyl}carbamoyl)-methyl]-2-methylamino-propionamide (Compound III); (S)—N—((S)-1-Cyclohexyl-2-{(S)-2-[5-(4-fluoro-phenoxy)-pyridin-3-yl]-pyrrolidin-1-yl}-2-oxo-ethyl)-2-methylamino-propionamide; and N-[1-Cyclohexyl-2-(2-(2-[(4-fluorophenyl)-methyl-amino]-pyridin-4-yl}pyrrolidin-1-yl)-2-oxo-ethyl]-2-methylamino-propinamide and pharmaceutically acceptable salts thereof.

Examples of other IAP inhibitors includes compounds disclosed in WO 05/097791 published on Oct. 20, 2005, which is hereby incorporated into the present application by reference. A preferred compound within the scope of formula (I) is N-[1-cyclohexyl-2-oxo-2-(6-phenethyl-octahydro-pyrrolo[2,3-c]pyridin-1-yl-ethyl]-2-methylamino-propionamide, hereinafter compound II.

Additional IAP inhibitors include compounds disclosed in WO 04/005284, PCT/US2006/013984, PCT/US2006/021850 all of which are hereby incorporated into the present application by reference.

Other IAP inhibitor compounds for use in the present invention include those disclosed in WO 06/069063, WO 05/069888, US2006/0014700, WO 04/007529, US2006/0025347, WO 06/010118, WO 05/069894, WO 06/017295, WO 04/007529, WO 05/094818.

In each case where citations of patent applications are given above, the subject matter relating to the compounds is hereby incorporated into the present application by reference. Comprised are likewise the pharmaceutically acceptable salts thereof, the corresponding racemates, diastereoisomers, enantiomers, tautomers, as well as the corresponding crystal modifications of above disclosed compounds where present, e.g., solvates, hydrates and polymorphs, which are disclosed therein. The compounds used as active ingredients in the combinations of the invention can be prepared and administered as described in the cited documents, respectively. Also within the scope of this invention is the combination of more than two separate active ingredients as set forth above, i.e., a pharmaceutical combination within the scope of this invention could include three active ingredients or more.

The terms “treatment” or “therapy” (especially of tyrosine protein kinase dependent diseases or disorders) refer to the prophylactic or preferably therapeutic (including but not limited to palliative, curing, symptom-alleviating, symptom-reducing, kinase-regulating and/or kinase-inhibiting) treatment of said diseases, especially of the diseases mentioned below.

A warm-blooded animal (or patient) is preferably a mammal, especially a human.

Where subsequently or above the term “use” is mentioned (as verb or noun) (relating to the use of an IAP inhibitor), this (if not indicated differently or suggested differently by the context) includes any one or more of the following embodiments of the invention, respectively (if not stated otherwise): the use in the treatment of a disease (especially diseases mediated or exacerbated by excessive TNF-α or characterized by constitutive TNF-α signaling), the use for the manufacture of pharmaceutical compositions for use in the treatment of diseases mediated or exacerbated by excessive TNF-α or characterized by constitutive TNF-α signaling, methods of use of one or more IAP inhibitors in the treatment of a disease mediated or exacerbated by excessive TNF-α or characterized by constitutive TNF-α signaling, pharmaceutical preparations comprising one or more IAP inhibitors for the treatment of said disease mediated or exacerbated by excessive TNF-α or characterized by constitutive TNF-α signaling, and one or more IAP inhibitors in the treatment of said disease mediated or exacerbated by excessive TNF-α or characterized by constitutive TNF-α signaling, as appropriate and expedient, if not stated otherwise. In particular, diseases to be treated and are thus preferred for “use” of an IAP inhibitor are selected from diseases that are mediated or exacerbated by excessive TNF-α or characterized by constitutive TNF-α signaling.

Preferred is the use of an IAP inhibitor in the therapy (including prophylaxis) of a proliferative disorder (especially which is characterized by constitutive TNF-α signaling.) selected from tumor or cancer diseases, especially against preferably a benign or especially malignant tumor or cancer disease, more preferably solid tumors, e.g. carcinoma of the brain, kidney, liver, adrenal gland, bladder, breast, stomach (especially gastric tumors), ovaries, colon, rectum, prostate, pancreas, lung (e.g. small or large cell lung carcinomas), vagina, thyroid, sarcoma, glioblastomas, multiple myeloma (MM) or gastrointestinal cancer, especially colon carcinoma or colorectal adenoma, or a tumor of the neck and head, e.g. squameous carcinoma of the head and neck, including neoplasias, especially of epithelial character, e.g. in the case of mammary carcinoma; an epidermal hyperproliferation (other than cancer), especially psoriasis; prostate hyperplasia; or a leukemia, especially acute myeloid leukemia (AML) and chronic myeloid leukemia (CML).

The precise dosage of an IAP inhibitor compound to be employed depends upon several factors including the host, the nature and the severity of the condition being treated, the mode of administration. The IAP inhibitor compound can be administered by any route including orally, parenterally, e.g., intraperitoneally, intravenously, intramuscularly, subcutaneously, intratumorally, or rectally, or enterally. Preferably the IAP inhibitor compound is administered orally, preferably at a daily dosage of 1-300 mg/kg body weight or, for most larger primates, a daily dosage of 50-5000, preferably 500-3000 mg. A preferred oral daily dosage is 1-75 mg/kg body weight or, for most larger primates, a daily dosage of 10-2000 mg, administered as a single dose or divided into multiple doses, such as twice daily dosing.

Usually, a small dose is administered initially and the dosage is gradually increased until the optimal dosage for the host under treatment is determined. The upper limit of dosage is that imposed by side effects and can be determined by trial for the host being treated.

Dosage regimens must be titrated to the particular indication, the age, weight, and general physical condition of the patient, and the response desired but generally doses will be from about 10 to about 500 mg/day as needed in single or multiple daily administration. In general, an initial treatment regimen can be copied from that known to be effective in interfering with TNF-α activity for other TNF-α mediated disease states by the compounds of the present invention. Treated individuals will be regularly checked for T cell numbers and T4/T8 ratios and/or measures of viremia such as levels of reverse transcriptase or viral proteins, and/or for progression of cytokine-mediated disease associated problems such as cachexia or muscle degeneration. If no effect is soon following the normal treatment regimen, then the amount of cytokine activity interfering agent administered is increased; e.g., by fifty percent a week.

IAP inhibitor compounds may be combined with one or more pharmaceutically acceptable carriers and, optionally, one or more other conventional pharmaceutical adjuvants and administered enterally, e.g. orally, in the form of tablets, capsules, caplets, etc. or parenterally, e.g., intraperitoneally or intravenously, in the form of sterile injectable solutions or suspensions. The enteral and parenteral compositions may be prepared by conventional means.

Production of TNF-α with an IAP inhibitor compound can be conveniently assayed using anti-TNF-α antibodies. For example, plates (Nunc Immunoplates, Roskilde, DK) are treated with 5 μg/mL of purified rabbit anti-TNF-α antibodies at 4° C. for 12 to 14 hours. The plates then are blocked for 2 hours at 25° C. with PBS/0.05% Tween containing 5 mg/mL BSA: After washing, 100 μL of unknowns as well as controls are applied and the plates incubated at 4° C. for 12 to 14. hours. The plates are washed and assayed with a conjugate of peroxidase (horseradish) and mouse anti-TNF-α monoclonal antibodies, and the color developed with o-phenylenediamine in phosphate-citrate buffer containing 0.012% hydrogen peroxide and read at 492 nm.

The following examples are offered by way of illustration and are not intended to limit the scope of the invention.

### Example 1

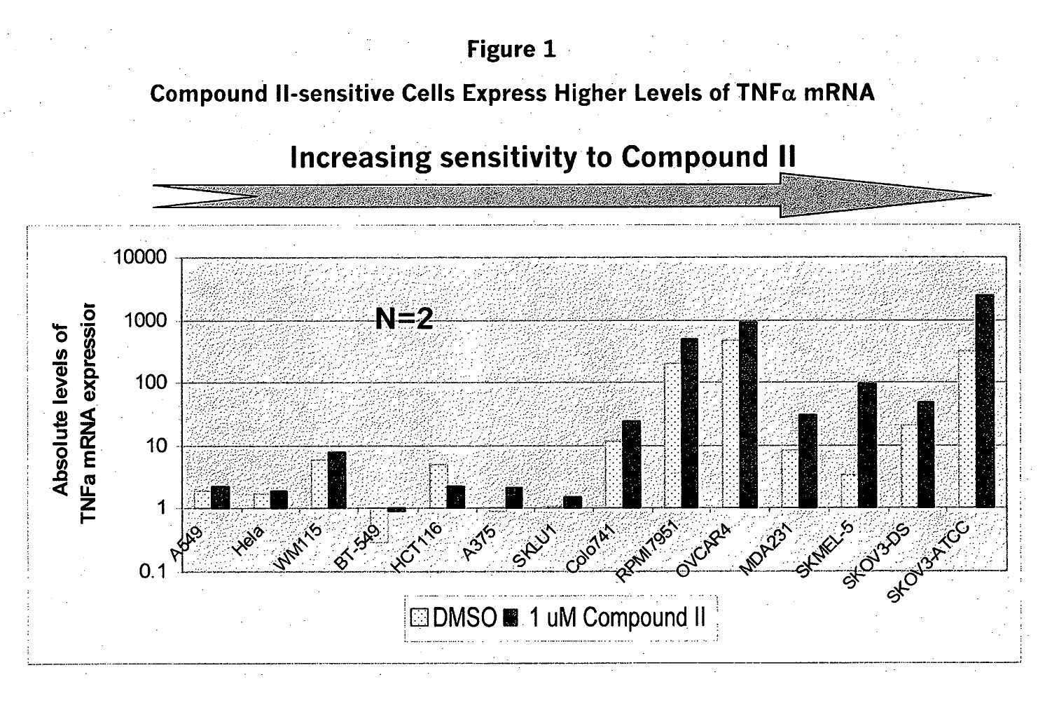

N-D-Cyclohexyl-2-(2-{2-[(4-fluorophenyl)-methyl-amino]-pyridin-4-yl}pyrrolidin-1-yl)-2-oxo-ethyl]-2-methylamino-propinamide, hereinafter Compound II, was tested in a number of cell lines as shown in FIG. 1. Each of the tumor cell lines indicated was treated for 18 hrs with 1 uM of the Compound II. mRNA was harvested using Qiagen's TurboCapture mRNA isolation kit. cDNA was synthesized using BioRad iScript cDNA synthesis kit. Primers specific for the cDNA encoding TNFalpha were then used to PCR amplify TNF cDNA from each sample using Applied. Biosystems TaqMan Universal PCR Master Mix. Data is normalized to B-Actin mRNA and expressed as relative levels of TNF mRNA.

FIG. 1 shows that sensitive cell lines (72 hour IC50<1 uM) express higher baseline levels of TNF mRNA and respond to Compound II treatment by increasing expression of TNF mRNA. Implicit in these findings is that TNF levels may be used to predict sensitivity to a Smac Mimetic compound and that assessment of rising TNF levels may have potential as a strategy for monitoring a therapeutic response.

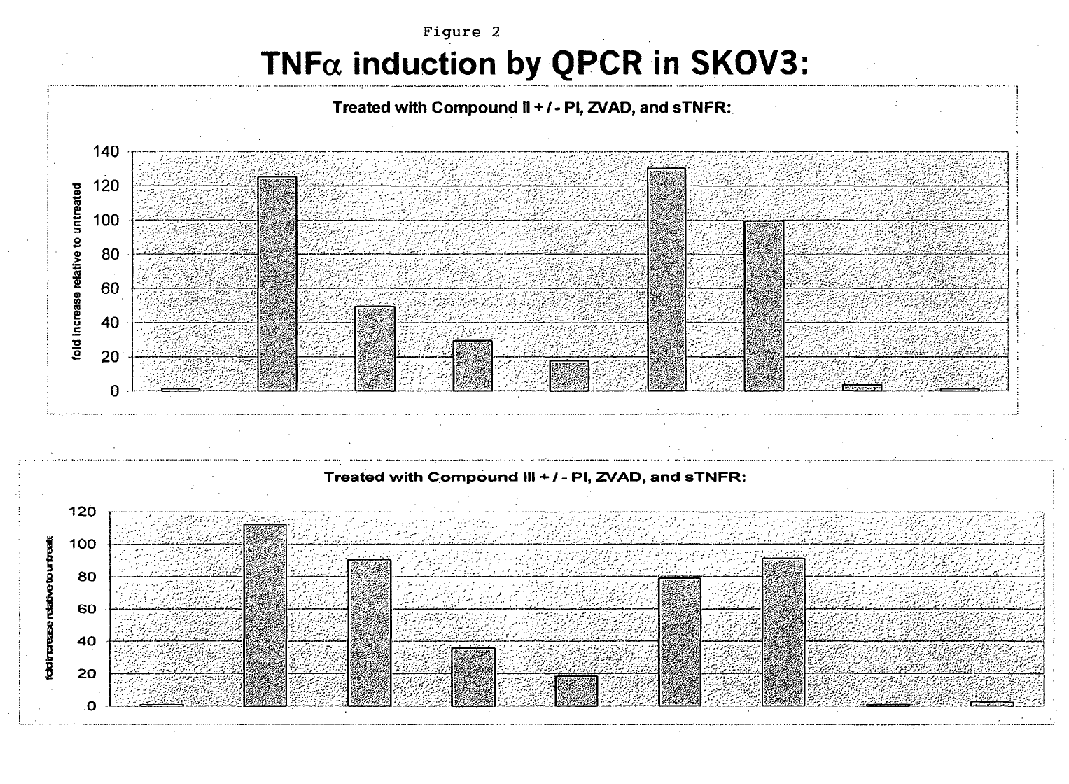

FIG. 2 shows how compounds II and III induce TNFα mRNA in SKOV-3 cells in a dose dependent manner. TNFα induction required proteosome activity since it is inhibited by MG132 (PI). TNFα induction does not require Caspase activity (is not blocked by ZVAD) but does require autocrine TNFα signaling since it is blocked with soluble TNFα receptor (STR).

The graph depicting TNF induction by compound II includes nine bars correlating to the fold increase of TNF relative to untreated cells. Reading from left to right, the first bar represents untreated cells (app. 0-1 fold). The second bar represents 1000 nM of compound II (app. 120-130 fold increase). The third bar represents 100 nM of compound II (app. 50 fold increase). The fourth bar represents 1000 nM of compound II+PI (app. 25-30 fold increase). The fifth bar represents 100 nM of compound II+PI (app. 15-20 fold increase). The sixth bar represents 1000 nM of compound II+ZVAD (app. 125-130 fold increase). The seventh bar represents 100 nM compound II+ZVAD (app. 95-100 fold increase). The eighth bar represents 1000 nM compound II+sTNFR (app. 0-5 fold increase). The ninth bar represents 100 nm compound II+sTNFR (app. 0-1).

The graph depicting TNF induction by compound III includes nine bars correlating to the fold increase of TNF relative to untreated cells. Reading from left to right, the first bar represents untreated cells (app. 0-1 fold). The second bar represents 1000 nM of compound III (app. 105-115 fold increase). The third bar represents 100 nM of compound III (app. 85-95 fold increase). The fourth bar represents 1000 nM of compound III+PI (app. 30-40 fold increase). The fifth bar represents 100 nM of compound III+PI (app. 15-20 fold increase). The sixth bar represents 1000 nM of compound III+ZVAD (app. 75-80 fold increase). The seventh bar represents 100 nM compound III+ZVAD (app. 85-95 fold increase). The eighth bar represents 1000 nM compound III+sTNFR (app. 0-1 fold increase). The ninth bar represents 100 nm compound III+sTNFR (app: 0-3 fold increase).

Variations, modification, and other implementations of what is described herein will occur to those of ordinary skill in the art without departing from the spirit and the essential characteristics of the present teachings. Accordingly the scope of the invention is to be defined not by the preceding illustrative description but instead by the following claims, and all changes that come within the meaning and range of equivalency of the claims are intended to be embraced therein.

1. A method to predict which patients will respond to a IAP inhibiting compound comprising:
a) administering an IAP inhibitor compound to a patient, and
b) measuring TNF-α and/or IL-8 levels.

2. A method according to claim 1 comprising the additional step of D) determining that the patient will be a non-responder if the correlation coefficient is less than ______.

3. The method of claim 1, wherein the IAP inhibiting compound has the structure of formula I: 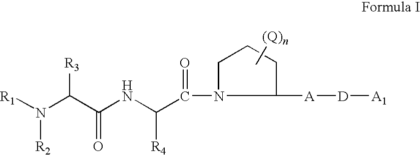
or pharmaceutically acceptable salts thereof, wherein
R₁ is H, C₁-C₄ alkyl, C₂-C₄ alkenyl, C₂-Cₐ alkynyl or C₃-C₁₀ cycloalkyl, which R₁ may be unsubstituted or substituted;
R₂ is H, C₁-C₄ alkyl, C₂-C₄ alkenyl, C₂-C₄ alkynyl, C₃-C₁₀ cycloalkyl which R₂ may be unsubstituted or substituted;
R₃ is H, CF₃, C₂F₅, C₁-C₄ alkyl, C₂-C₄ alkenyl, C₂-C₄ alkynyl, CH₂-Z or R₂ and R₃ taken together with the nitrogen atom to which they are attached form a heterocyclic ring, which alkyl, alkenyl, alkynyl or het ring may be unsubstituted or substituted;
Z is H, OH, F, Cl, CH₃, CH₂Cl, CH₂F or CH₂OH;
R₄ is C₀₋₁₀ alkyl, C₃-C₁₀ cycloalkyl, wherein the C₀₋₁₀ alkyl, or cycloalkyl group is unsubstituted or substituted;
A is het, which may be substituted or unsubstituted;
D is C₁-C₇ alkylene or O₂—C₉ alkenylene, C(O), O, NR₇, S(O)ᵣ, C(O)—C₁-C₁₀ alkyl, O—C₁-C₁₀ alkyl, S(O)ᵣ—C₁-C_{1c}, alkyl, C(O)C₀-C₁₀ arylalkyl CO₀-C₁₀ arylalkyl, or S(O)r C₀-C₁₀ arylalkyl, which alkyl and aryl groups may be unsubstituted or substituted;
r is 0, 1, or 2;
A₁ is a substituted aryl or unsubstituted or substituted het which substituents on aryl and het are halo, lower alkoxy, NR₅R₆, CN, NO₂ or SR₅;
each Q is independently H, C₁-C₁₀ alkyl, C₁-C₁₀ alkoxy, aryl C₁-C₁₀ alkoxy, OH, O—C₁-C₁₀-alkyl, (CH₂)₀₋₆—C₃-C₇ cycloalkyl, aryl, aryl C₁-C₁, alkyl, O—(CH₂)₀₋₆aryl, (CH₂)₁₋₆het, het, O—(CH₂)₁₋₆het, —OR₁₁, C(O)R₁₁, —C(O)N(R₁₁)(R₁₂), N(R₁₁)(R₁₂)SR₁₁, S(O)R₁₁, S(O)₂ R₁₁, S(O)₂—N(R₁₁)(R₁₂), or NR₁₁—S(O)₂—(R₁₂), wherein alkyl, cycloalkyl and aryl are unsubstituted or substituted;
n is 0, 1, 2 or 3, 4, 5, 6 or 7;
het is a 5-7 membered monocyclic heterocyclic ring containing 1-4 heteroring atoms selected from N, O and S or an 8-12 membered fused ring system that includes one 5-7 membered monocyclic heterocyclic ring containing 1, 2, or 3 heteroring atoms selected from N, O and S, which het is unsubstituted or substituted;
R₁₁ and R₁₂ are independently H, C₁-C₁₀ alkyl, (CH₂)₀₄—C₃-C₇cycloalkyl, (CH₂)₀₋₆—(CH)₀₋₁(aryl)₁₋₂, C(O)—C₁-C₁₀alkyl, —C(O)—(CH₂)₁₄—C₃-C₇cycloalkyl, —C(O)—O—(CH₂)₀₋₆-aryl, —C(O)—(CH₂)₀₋₆—O-fluorenyl, C(O)—NH—(CH₂)₀₋₄-aryl, C(O)—(CH₂)₀₋₄-aryl, C(O)—(CH₂)₁₋₆-het, —C(S)—C₁-C₁₀alkyl, —C(S)—(CH₂)₁₆—C₃-C₇cycloalkyl, —C(S)—O—(CH₂)₀₋₆-aryl, —C(S)—(CH₂)₀₋₆—O-fluorenyl, C(S)—NH—(CH₂)₀₋₆-aryl, —C(S)—(CH₂)₀₋₃-aryl or C(S)—(CH₂)₁₋₆-het, C(O)R₁₁, C(O)NR₁₁R₁₂, C(O)OR₁₁, S(O)nR₁₁, S(O)mNR₁₁R₁₂, m=1 or 2, C(S)R₁₁, C(S)NR₁₁R₁₂, C(S)OR₁₁, wherein alkyl, cycloalkyl and aryl are unsubstituted or substituted; or R₁₁ and R₁₂ are a substituent that facilitates transport of the molecule across a cell membrane; or R₁₁ and R₁₂ together with the nitrogen atom form het;
wherein the alkyl substituents of R₁₁ and R₁₂ may be unsubstituted or substituted by one or more substituents selected from C₁-C₁₀alkyl, halogen, OH, O—C₁-C₆alkyl, —S—C₁-C₆alkyl, CF₃ or NR₁R₁₂;
substituted cycloalkyl substituents of R₁₁ and R₁₂ are substituted by one or more substituents selected from a C₂-C₁₀ alkene; C₁-C₆alkyl; halogen; OH; O—C₁-C₆alkyl; S—C₁-C₆alkyl, CF₃; or NR₁₁R₁₂ and
substituted het or substituted aryl of R₁₁ and R₁₂ are substituted by one or more substituents selected from halogen, hydroxy, C₁-Cₐ alkyl, C₁-C₄ alkoxy, nitro, CN O—C(O)—C₁-C₄alkyl and C(O)—O—C₁-C₄-alkyl;
R₅, R₆ and R₇ are independently hydrogen, lower alkyl, aryl, aryl lower alkyl, cycloalkyl, or cycloalkyl lower alkyl, and
wherein the substituents on R₁, R₂, R₃, R₄, Q, and A and A₁ groups are independently halo, hydroxy, lower alkyl, lower alkenyl, lower alkynyl, lower alkanoyl, lower alkoxy, aryl, aryl lower alkyl, amino, amino lower alkyl, diloweralkylamino, lower alkanoyl, amino lower alkoxy, nitro, cyano, cyano lower alkyl, carboxy, lower carbalkoxy, lower alkanoyl, aryloyl, lower arylalkanoyl, carbamoyl, N-mono- or N,N-dilower alkyl carbamoyl, lower alkyl carbamic acid ester, amidino, guanidine, ureido, mercapto, sulfo, lower alkylthio, sulfoamino, sulfonamide, benzosulfonamide, sulfonate, sulfanyl lower alkyl, aryl sulfonamide, halogen substituted aryl sulfonate, lower alkylsulfinyl, arylsulfinyl; aryl-lower alkylsulfinyl, lower alkylarylsulfinyl, lower alkylsulfonyl, arylsulfonyl, aryl-lower alkylsulfonyl, lower aryl alkyl lower alkylarylsulfonyl, halogen-lower alkylmercapto, halogen-lower alkylsulfonyl, phosphono (—P(═O)(OH)₂), hydroxy-lower alkoxy phosphoryl or di-lower alkoxyphosphoryl, (R₆)NC(O)—NR₁₀R₁₃, lower alkyl carbamic acid ester or carbamates or —NR₈R₁₄, wherein R₈ and R₁₄ can be the same or different and are independently H or lower alkyl, or R₈ and R₁₄ together with the N atom form a 3- to 8-membered heterocyclic ring containing a nitrogen heteroring atoms and may optionally contain one or two additional heteroring atoms selected from nitrogen, oxygen and sulfur, which heterocyclic ring may be unsubstituted or substituted with lower alkyl, halo, lower alkenyl, lower alkynyl, hydroxy, lower alkoxy, nitro, amino, lower alkyl, amino, diloweralkyl amino, cyano, carboxy, lower carbalkoxy, formyl, lower alkanoyl, oxo, carbarmoyl, N-lower or N,N-dilower alkyl carbamoyl, mercapto, or lower alkylthio, and
R₉, R₁₀, and R₁₃ are independently hydrogen, lower alkyl, halogen substituted lower alkyl, aryl, aryl lower alkyl, halogen substituted aryl, halogen substituted aryl lower alkyl.

4. A method for determining the responsiveness of an individual with a disease characterized by constitutive TNF-α signaling to treatment with a IAP inhibiting compound comprising:
a) administering an IAP inhibitor compound to a patient, and
b) measuring TNF-α or IL-8 levels.

5. A method for treating diseases characterized by constitutive TNF-α signaling comprising:
a) administering an IAP inhibitor compound, and
b) measuring TNF-α levels.

6. The method of claim 4, wherein the IAP inhibiting compound has the structure of formula I: 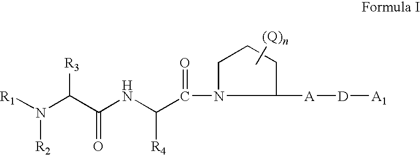
or pharmaceutically acceptable salts thereof, wherein
R₁ is H, C₁-C₄ alkyl, C₂-C₄ alkenyl, alkynyl or C₃-C₁₀ cycloalkyl, which R₁ may be unsubstituted or substituted;
R₂ is H, C₁-C₄ alkyl, C₂-C₄ alkenyl, C₂-C₄ alkynyl, C₃-C₁₀ cycloalkyl which R₂ may be unsubstituted or substituted;
R₃ is H, CF₃, C₂F₅, C₁-C₄ alkyl, C₂-C₄ alkenyl, C₂-C₄ alkynyl, CH₂-Z or R₂ and R₃ taken together with the nitrogen atom to which they are attached form a heterocyclic ring, which alkyl, alkenyl, alkynyl or het ring may be unsubstituted or substituted;
Z is H, OH, F, Cl, CH₃, CH₂Cl, CH₂F or CH₂OH;
R₄ is C₀₋₁₀ alkyl, C₃-C₁₀ cycloalkyl, wherein the C₀₋₁₀ alkyl, or cycloalkyl group is unsubstituted or substituted;
A is het, which may be substituted or unsubstituted;
D is C₁-C₇ alkylene or C₂-C₉ alkenylene, C(O), O, NR₇, S(O)ᵣ, C(O)—C₁-C₁₀ alkyl, O—C₁-C₁₀ alkyl, S(O)ᵣ—C₁-C₁₀ alkyl, C(O)C₀-C₁₀ arylalkyl C₀-C₁₀ arylalkyl, or S(O)ᵣ arylalkyl, which alkyl and aryl groups may be unsubstituted or substituted;
r is 0, 1, or 2;
A₁ is a substituted aryl or unsubstituted or substituted het which substituents on aryl and het are halo, lower alkoxy, NR₅R₆, CN, NO₂ or SR₅;
each Q is independently H, C₁-C₁₀ alkyl, C₁-C₁₀ alkoxy, aryl C₁-C₁₀ alkoxy, OH, O—C₁-C₁₀-alkyl, (CH₂)₀₋₆—C₃-C₂ cycloalkyl, aryl, aryl C₁-C₁₀ alkyl, O—(CH₀₋₆ aryl, (CH₂)₁₋₆het, het, O—(CH₂)₁₋₆het, —OR₁₁, C(O)R₁₁, —C(O)N(R₁₁)(R₁₂). N(R₁₁)(R₁₂), SR₁₁, S(O)R₁₁, S(O)₂ R₁₁, S(O)₂—N(R₁₁)(R₁₂), or NR₁₁—S(O)₂—(R₁₂), wherein alkyl, cycloalkyl and aryl are unsubstituted or substituted;
n is 0, 1, 2 or 3, 4, 5, 6 or 7;
het is a 5-7 membered monocyclic heterocyclic ring containing 1-4 heteroring atoms selected from N, O and S or an 8-12 membered fused ring system that includes one 5-7 membered monocyclic heterocyclic ring containing 1, 2, or 3 heteroring atoms selected from N, O and S, which het is unsubstituted or substituted;
R₁₁ and R₁₂ are independently H, C₁-C₁₀ alkyl, (CH₂)₀₋₆—C₃-C₂cycloalkyl, (CH₂)ₐₛ—(CH)₀₋₁(aryl)₁₄, C(O)—C₁-C₁₀alkyl, —C(O)—(CH₂)₁₋₆—C₃-C₇cycloalkyl, —C(O)—O—(CH₂)₀₋₆-aryl, —C(O)—(CH₂)₀₋₆—O-fluorenyl, C(O)—NH—(CH₂)₀₋₆-aryl, C(O)—(CH₂)₀₋₆-aryl, C(O)—(CH₂)₁₋₆-het, —C(S)—C₁-C₁₀alkyl, —C(S)—(CH₂)₁₋₆—C₃-C₇cycloalkyl, —C(S)—O—(CH₂)₀₋₆-aryl, —C(S)—(CH₂)₀₋₆—O-fluorenyl, C(S)—NH—(CH₂)₀₋₆-aryl, —C(S)—(CH₂)₀₋₆-aryl or C(S)—(CH₂)₁₋₆-het, C(O)R₁₁, C(O)NR₁₁R₁₂, C(O)OR₁₁, S(O)nR₁₁, S(O)mNR₁₁R₁₂, m=1 or 2, C(S)R₁₁, C(S)NR₁₁R₁₂, C(S)OR₁₁, wherein alkyl, cycloalkyl and aryl are unsubstituted or substituted; or R₁₁ and R₁₂ are a substituent that facilitates transport of the molecule across a cell membrane; or R₁₁ and R₁₂ together with the nitrogen atom form het;
wherein the alkyl substituents of R₁₁ and R₁₂ may be unsubstituted or substituted by one or more substituents selected from C₁-C₁₀alkyl, halogen, OH, O—C₁-C₆alkyl, —S—C₁-C₆alkyl, CF₃ or NR₁₁R₁₂;
substituted cycloalkyl substituents of R₁₁ and R₁₂ are substituted by one or more substituents selected from a C₂-C₁₀ alkene; C₁-C₆alkyl; halogen; OH; O—C₁-C₆alkyl; S—C₁-C₆alkyl, CF₃; or NR₁₁R₁₂ and
substituted het or substituted aryl of R₁₁ and R₁₂ are substituted by one or more substituents selected from halogen, hydroxy, C₁-C₄ alkyl, C₁-C₄ alkoxy, nitro, CN O—C(O)—C₁-C₄alkyl and C(O)—O—C₁-C₄-alkyl;
R₅, R₆ and R₇ are independently hydrogen, lower alkyl, aryl, aryl lower alkyl, cycloalkyl, or cycloalkyl lower alkyl, and
wherein the substituents on R₁, R₂, R₃, R₄, Q, and A and A₁ groups are independently halo, hydroxy, lower alkyl, lower alkenyl, lower alkynyl, lower alkanoyl, lower alkoxy, aryl, aryl lower alkyl, amino, amino lower alkyl, diloweralkylamino, lower alkanoyl, amino lower alkoxy, nitro, cyano, cyano lower alkyl, carboxy, lower carbalkoxy, lower alkanoyl, aryloyl, lower arylalkanoyl, carbamoyl, N-mono- or N,N-diloweralkyl carbamoyl, lower alkyl carbamic acid ester, amidino, guanidine, ureido, mercapto, sulfo, lower alkylthio, sulfoamino, sulfonamide, benzosulfonamide, sulfonate, sulfanyl lower alkyl, aryl sulfonamide, halogen substituted aryl sulfonate, lower alkylsulfinyl, arylsulfinyl; aryl-lower alkylsulfinyl, lower alkylarylsulfinyl, lower alkylsulfonyl, arylsulfonyl, aryl-lower alkylsulfonyl, lower aryl alkyl lower alkylarylsulfonyl, halogen-lower alkylmercapto, halogen-lower alkylsulfonyl, phosphono (—P(═O)(OH)₂), hydroxy-lower alkoxy phosphoryl or di-lower alkoxyphosphoryl, (R₉)NC(O)—NR₁₀R₁₃, lower alkyl carbamic acid ester or carbamates or —NR₈R₁₄, wherein R₈ and R₁₄ can be the same or different and are independently H or lower alkyl, or R₈ and R₁₄ together with the N atom form a 3- to 8-membered heterocyclic ring containing a nitrogen heteroring atoms and may optionally contain one or two addition heteroring atoms selected from nitrogen, oxygen and sulfur, which heterocyclic ring may be unsubstituted or substituted with lower alkyl, halo, lower alkenyl, lower alkynyl, hydroxy, lower alkoxy, nitro, amino, lower alkyl, amino, diloweralkyl amino, cyano, carboxy, lower carbalkoxy, formyl, lower alkanoyl, oxo, carbarmoyl, N-lower or N,N-dilower alkyl carbamoyl, mercapto, or lower alkylthio, and
R₉, R₁₀, and R₁₃ are independently hydrogen, lower alkyl, halogen substituted lower alkyl, aryl, aryl lower alkyl, halogen substituted aryl, halogen substituted aryl lower alkyl.

17. A method according to claim 1, wherein where the IAP inhibitor compound is selected from N-1-Cyclohexyl-2-{2-[4-(4-fluoro-benzoyl)-thiazol-2-yl]-pyrrolidin-1-yl)-2-oxo-ethyl)-2-methylamino-propionamide; N-[Cyclohexyl-(ethyl-{1-[5-(4-fluoro-benzoyl)-pyridin-3-yl]-propyt}carbamoyl)-methyl]-2-methylamino-propionamide; N-(1-Cyclohexyl-2-{2-[5-(4-fluoro-phenoxy)-pyridin-3-yl]-pyrrolidin-1-yl)-2-oxo-ethyl)-2-methylamino-propionamide; and N-[1-Cyclohexyl-2-(2-{2-[(4-fluorophenyl)-methyl-amino]-pyridin-4-yl)pyrrolidin-1-yl)-2-oxo-ethyl]-2-methylamino-propinamide and pharmaceutically acceptable salts thereof.

8. Use of IAP inhibitor compounds in the treatment of proliferative diseases characterized by constitutive TNF-α signaling.

9. Use of a compound of the formula I, or an N-oxide or pharmaceutically acceptable salt thereof, in the treatment of a disease characterized by constitutive TNF-α signaling wherein the compound of formula I has the following structure: 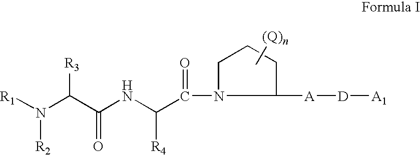
or pharmaceutically acceptable salts thereof, wherein
R₁ is H, C₁-C₄ alkyl, C₂-C₄ alkenyl, C₂-C₄ alkynyl or C₃-C₁₀ cycloalkyl, which R₁ may be unsubstituted or substituted;
R₂ is H, C₁-C₄ alkyl, C₂-C₄ alkenyl, C₂-C₄ alkynyl, C₃-C₁₀ cycloalkyl which R₂ may be unsubstituted or substituted;
R₃ is H, CF₃, C₂F₅, C₁-C₄ alkyl, C₂-C₄ alkenyl, C₂-C₄ alkynyl, CH₂—Z or R₂ and R₃ taken together with the nitrogen atom to which they are attached form a heterocyclic ring, which alkyl, alkenyl, alkynyl or het ring may be unsubstituted or substituted;
Z is H, OH, F, Cl, CH₃, CH₂Cl, CH₂F or CH₂OH;
R₄ is C₀₋₁₀ alkyl, C₃-C₁₀ cycloalkyl, wherein the C₀₋₁₀ alkyl, or cycloalkyl group is unsubstituted or substituted;
A is het, which may be substituted or unsubstituted;
D is C₁-C₇ alkylene or C₂-C₉ alkenylene; C(O), O, NR₇, S(O)ᵣ, C(O)—C₁-C₁₀ alkyl, O—C₁-C₁₀ alkyl, S(O)r-C₁-C₁₀ alkyl, C(O)C₀-C₁₀ arylalkyl CH₀—C₁₀ arylalkyl, or S(O)ᵣ C₀-C₁₀ arylalkyl, which alkyl and aryl groups may be unsubstituted or substituted;
r is 0, 1, or 2;
A₁ is a substituted aryl or unsubstituted or substituted het which substituents on aryl and het are halo, lower alkoxy, NR₅R₆, CN, NO₂ or SR₅;
each Q is independently H, C₁-C₁₀ alkyl, C₁-C₁₀ alkoxy, aryl C₁-C₁₀ alkoxy, OH, O—C₁-C₁₀-alkyl, (CH₂)₀₋₆—C₃-C₇ cycloalkyl, aryl, aryl C₁-C₁₀ alkyl, O—(CH₂)₀₋₆ aryl, (CH₂)₁₋₆het, het, O—(CH₂)₁₋₆het, —OR₁₁, C(O)R₁₁, —C(O)N(R₁₁)(R₁₂), N(R₁₁)(R₁₂), SR₁₁, S(O)R₁₇, S(O)₂ R₁₁, S(O)₂—N(R₁₁)(R₁₂), or NR₁₁—S(O)ᵣ(R₁₂), wherein alkyl, cycloalkyl and aryl are unsubstituted or substituted;
n is 0, 1, 2 or 3, 4, 5, 6 or 7;
het is a 5-7 membered monocyclic heterocyclic ring containing 1-4 heteroring atoms selected from N, O and S or an 8-12 membered fused ring system that includes one 5-7 membered monocyclic heterocyclic ring containing 1, 2, or 3 heteroring atoms selected from N, O and S, which het is unsubstituted or substituted;
R₁₁ and R₁₂ are independently H, C₁-C₁₀ alkyl, (CH₂)₀₋₆—C₃-C₇cycloalkyl, (CH₂)₀₋₆—(CH)₀₋₁(aryl)₁₋₂, C(O)—C₁-C₁₀alkyl, —C(O)—(CH₂)₁₄₅—C₃-C₇cycloalkyl, —C(O)—O—(CH₂)₀₋₆-aryl, —C(O)—(CH₂)₀₋₆—O-fluorenyl, C(O)—NH—(OH₂)₀₋₆-aryl, C(O)—(CH₂)₀₋₆-aryl, C(O)—(OH₂)₁₋₆-het, —C(S)—C₁-C₁₀alkyl, —C(S)—(CH₂)₁₋₆—C₃-C₇cycloalkyl, —C(S)—O—(CH₂)₁₋₆-aryl, —C(S)—(CH₂)₀₋₆—O-fluorenyl, C(S)—NH—(CH₂)₀₋₆-aryl, —C(S)—(CH₂)₀₋₆-aryl or C(S)—(OH₂)₁₋₆-het, C(O)R₁₁, C(O)NR₁₁R₁₂, C(O)OR₁₁, S(O)nR₁₁, S(O)mNR₁₁R₁₂, m=1 or 2, C(S)R₁₁, C(S)NR₁₁R₁₂, C(S)OR₁₁, wherein alkyl, cycloalkyl and aryl are unsubstituted or substituted; or R₁₁ and R₁₂ are a substituent that facilitates transport of the molecule across a cell membrane; or R₁₁ and R₁₂ together with the nitrogen atom form het;
wherein the alkyl substituents of R₁₁ and R₁₂ may be unsubstituted or substituted by one or more substituents selected from C₁-C₁₀alkyl, halogen, OH, O—C₁-C₆alkyl, —S—C₁-C₆alkyl, CF₃ or NR₁₁R₁₂:
substituted cycloalkyl substituents of R₁₁ and R₁₂ are substituted by one or more substituents selected from a C₂-C₁₀alkene; C₁-C₆alkyl; halogen; OH; O—C₁-C₆alkyl; S—C₁-C₆alkyl, CF₃; or NR₁₁R₁₂ and
substituted het or substituted aryl of R₁₁ and R₁₂ are substituted by one or more substituents selected from halogen, hydroxy, C₁-C₄ alkyl, C₁-C₄ alkoxy, nitro, CN O—C(O)—C₁-C₄alkyl and C(O)—O—C₁-C₄-alkyl;
R₅, R₆ and R₇ are independently hydrogen, lower alkyl, aryl, aryl lower alkyl, cycloalkyl, or cycloalkyl lower alkyl, and
wherein the substituents on R₁, R₂, R₃, R₄, Q, and A and A₁ groups are independently halo, hydroxy, lower alkyl, lower alkenyl, lower alkynyl, lower alkanoyl, lower alkoxy, aryl, aryl lower alkyl, amino, amino lower alkyl, diloweralkylamino, lower alkanoyl, amino lower alkoxy, nitro, cyano, cyano lower alkyl, carboxy, lower carbalkoxy, lower alkanoyl, aryloyl, lower arylalkanoyl, carbamoyl, N-mono- or N,N-dilower alkyl carbamoyl, lower alkyl carbamic acid ester, amidino, guanidine, ureido, mercapto, sulfo, lower alkylthio, sulfoamino, sulfonamide, benzosulfonamide, sulfonate, sulfanyl lower alkyl, aryl sulfonamide, halogen substituted aryl sulfonate, lower alkylsulfinyl, arylsulfinyl; aryl-lower alkylsulfinyl, lower alkylarylsuffinyl, lower alkylsulfonyl, arylsulfonyl, aryl-lower alkylsulfonyl, lower aryl alkyl lower alkylarylsulfonyl, halogen-lower alkylmercapto, halogen-lower alkylsulfonyl, phosphono (—P(═O)(OH)₂), hydroxy-lower alkoxy phosphoryl or di-lower alkoxyphosphoryl, (R₉)NC(O)—NR₁₀R₁₃, lower alkyl carbamic acid ester or carbamates or —NR₈R₁₄, wherein R₈ and R₁₄ can be the same or different and are independently H or lower alkyl, or R₈ and R₁₄ together with the N atom form a 3- to 8-membered heterocyclic ring containing a nitrogen heteroring atom's and may optionally contain one or two additional heteroring atoms selected from nitrogen, oxygen and sulfur, which heterocyclic ring may be unsubstituted or substituted with lower alkyl, halo, lower alkenyl, lower alkynyl, hydroxy, lower alkoxy, nitro, amino, lower alkyl, amino, diloweralkyl amino, cyano, carboxy, lower carbalkoxy, formyl, lower alkanoyl, oxo, carbarmoyl, N-lower or N,N-dilower alkyl carbamoyl, mercapto, or lower alkylthio, and
R₉, R₁₀, and R₁₃ are independently hydrogen, lower alkyl, halogen substituted lower alkyl, aryl, aryl lower alkyl, halogen substituted aryl, halogen substituted aryl lower alkyl.

10. Use of a compound of the formula I, according to claim 9, or a pharmaceutically acceptable salt thereof, for the manufacture of a pharmaceutical composition for the treatment of a disease characterized by constitutive TNF-α signaling.

11. A method of treatment a disease characterized by constitutive TNF-α signaling, comprising administering to a warm-blooded animal, especially a human, in need of such treatment a pharmaceutically effective amount of a compound of the formula I, or a pharmaceutically acceptable salt thereof, according to claim 9.

12. A use according to claim 9 where the compound of formula I is selected from N-1-Cyclohexyl-2-{2-(4-(4-fluoro-benzoyl)-thiazol-2-yl]-pyrrolidin-1-yl}-2-oxo-ethyl)-2-methylamino-propionamide; N-[Cyclohexyl-(ethyl-{1-[5-(4-fluoro-benzoyl)-pyridin-3-yl)-propyl)carbamoyl)-methyl]-2-methylamino-propionamide; N-(1-cyclophenyl-2-{2-(5-(4-fluoro-phenoq)-pyridin-3-yl]-pyrrolidin-1-yl}-2-oxo-ethyl)-2-methylamino-propionamide; and N-(1-Cyclohexyl-2-(2-{2-[(4-fluorophenyl)-methyl-amino]-pyridin-4-yl}pyrrolidin-1-yl)-2-oxo-ethyl]-2-methylamino-propinamide and pharmaceutically acceptable salts thereof.

13. A use according to claim 10 where the compound of formula I is selected from N-(1-Cyclohexyl-2-{2-(4-(4-fluoro-benzoyl)-thiazol-2-yl]-pyrrolidin-1-yl}-2-oxo-ethyl)-2-methylamino-propionamide; N-(Cyclohexyl-(ethyl-{1-(5-(4-fluoro-benzoyl)-pyridin-3-yl]-propyl}carbamoyl)-methyl]-2-methylamino-propionamide; N-(1-Cyclohexyl-2-{2-[5-(4-fluoro-phenoxy)-pyridin-3-yl]-pyrrolidin-1-yl}-2-oxo-ethyl)-2-methylamino-propionamide; and N-(1-Cyclohexyl-2-(2-{2-[(4-fluorophenyl)-methyl-amino]-pyridin-4-yl}pyrrolidin-1-yl)-2-oxo-ethyl]-2-methylamino-propinamide and pharmaceutically acceptable salts thereof.

14. A method according to claim 11 where the compound of formula I is selected from N-(1-Cyclohexyl-2-{2-[4-(4-fluoro-benzoyl)-thiazol-2-yl]-pyrrolidin-1-yl}-2-oxo-ethyl)-2-methylamino-propionamide; N-[Cyclohexyl-(ethyl-{1-[5-(4-fluoro-benzoyl)-pyridin-3-yl]-propyl}carbamoyl)-methyl-2-methylamino-propionamide; N-(1-Cyclohexyl-2-{2-[5-(4-fluoro-phenoxy)-pyridin-3-yl)-pyrrolidin-1-yl}-2-oxo-ethyl)-2-methylamino-propionamide; and N-[1-Cyclohexyl-2-(2-{2-[(4-fluorophenyl)-methyl-amino]-pyridin-4-yl}pyrrolidin-1-yl)-2-oxo-ethyl]-2-methylamino-propinamide and pharmaceutically acceptable salts thereof.

15. A use according to claim 9 wherein the disease is a proliferative disease.

16. A use according to claim 9 wherein the disease is a selected from cancers, such as solid tumors and blood-born tumors; heart disease, such as congestive heart failure; and viral, genetic, inflammatory, allergic, and autoimmune diseases.

17. The method of claim 5, wherein the IAP inhibiting compound has the structure of formula I: 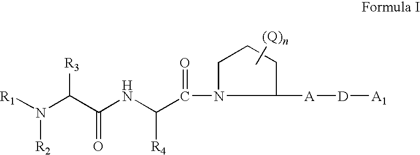
or pharmaceutically acceptable salts thereof, wherein
R₁ is H, C₁-C₄ alkyl, C₂-C₄ alkenyl, C₂-C₄ alkynyl or cycloalkyl, which R₁ may be unsubstituted or substituted;
R₂ is H, C₁-C₄ alkyl, C₂-C₄ alkenyl, C₂-C₄ alkynyl, C₃-C₁₀ cycloalkyl which R₂ may be unsubstituted or substituted;
R₃ is H, CF₃, C₂F₅, C₁-C₄ alkyl, C₂-C₄ alkenyl, C₂-C₄ alkynyl, CH₂-Z or R₂ and R₃ taken together with the nitrogen atom to which they are attached form a heterocyclic ring, which alkyl, alkenyl, alkynyl or het ring may be unsubstituted or substituted;
Z is H, OH, F, Cl, CH₃, CH₂Cl, CH₂F or CH₂OH;
R₄ is C₀₋₁₀ alkyl, C₃-C₁₀ cycloalkyl, wherein the C₀₋₁₀ alkyl, or cycloalkyl group is unsubstituted or substituted;
A is het, which may be substituted or unsubstituted;
D is C₁-C₇ alkylene or C₂-C₉ alkenylene, C(O), O, NR₇, S(O)ᵣ, C(O)—C₁-C₁₀ alkyl, O—C₁-C₁₀ alkyl, S(O)ᵣ—C₁-C₁₀ alkyl, C(O)C₀-C₁₀ arylalkyl C₀-C₁₀ arylalkyl, or S(O)r O₃—C₁₀ arylalkyl, which alkyl and aryl groups may be unsubstituted or substituted;
r is 0, 1, or 2;
A₁ is a substituted aryl or unsubstituted or substituted het which substituents on aryl and het are halo, lower alkoxy, NR₅R₆, CN, NO₂ or SR₅;
each Q is independently H, C₁-C₁₀ alkyl, O₁—C₁₀ alkoxy, aryl C₁-C_{1D} alkoxy, OH, O—C₁-C₁₀-alkyl, (CH₂)₀₋₆—C₃-C₇ cycloalkyl, aryl, aryl C₁-C₁₀ alkyl, O—(CH₂)₀₋₆ aryl, (CH₂)₁₋₆het, het, O—(CH₂)₁₋₆het, —OR₁₁, C(O)R₁₁, —C(O)N(R₁₁)(R₁₂), N(R₁₁)(R₁₂), SR₁₁, S(O)R₁₁, S(O)₂ R₁₁, S(O)₂—N(R₁₁)(R₁₂), or NR₁₁—S(O)₂—(R₁₂), wherein alkyl, cycloalkyl and aryl are unsubstituted or substituted;
n is 0, 1, 2 or 3, 4, 5, 6 or 7;
het is a 5-7 membered monocyclic heterocyclic ring containing 1-4 heteroring atoms selected from N, O and S or an 8-12 membered fused ring system that includes one 5-7 membered monocyclic heterocyclic ring containing 1, 2, or 3 heteroring atoms selected from N, O and S, which het is unsubstituted or substituted;
R₁₁ and R₁₂ are independently H, C₁-C₁₀ alkyl, (CH₂)₀₋₆—C₃-C₇cycloalkyl, (CH₂)₀₋₆—(CH)₀₋₁(aryl)₁₋₂, C(O)—C₁-C₁₀alkyl, —C(O)—(CH₂)₁₋₆—C₃-C₇cycloalkyl, —C(O)—O—(CH₂)₀₋₆-aryl, —O(O) —(CH₂)₀₋₆—O-fluorenyl, C(O)—NH—(CH₂)₀₋₆-aryl, C(O)—(CH₂)₀₋₆-aryl, C(O)—(CH₂)₁₋₆-het, —C(S)—C₁-C₁₀alkyl, —C(S)—(CH₂)₁₋₆—C₃-C₇cycloalkyl, —C(S)—O—(CH₂)₀₋₆-aryl, —C(S)—(CH₂)₀₋₆—O-fluorenyl, C(S)—NH—(CH₂)₀₋₆-aryl, —C(S)—(CH₂)₁₋₆-aryl or C(S)—(CH₂)₁₋₆-het, C(O)R₁₁, C(O)NR₁₁R₁₂, C(O)OR₁₁, S(O)nR₁₁, S(O)mNR₁₁R₁₂, m=1 or 2, C(S)R₁₁, C(S)NR₁₁R₁₂, C(S)OR₁₁, wherein alkyl, cycloalkyl and aryl are unsubstituted or substituted; or R₁₁ and R₁₂ are a substituent that facilitates transport of the molecule across a cell membrane; or R₁₁ and R₁₂ together with the nitrogen atom form het;
wherein the alkyl substituents of R₁₁ and R₁₂ may be unsubstituted or substituted by one or more substituents selected from C₁-C₁₀alkyl, halogen, OH, O—C₁-C₆alkyl, —S—C₁-C₆alkyl, CF₃ or NR₁₁R₁₂:
substituted cycloalkyl substituents of R₁₁ and R₁₂ are substituted by one or more substituents selected from a C₂-C₁₀ alkene; C₁-C₆alkyl; halogen; OH; O—C₁-C₆alkyl; S—C₁-C₆alkyl, CF₃; or NR₁₁R₁₂ and
substituted het or substituted aryl of R₁₁ and R₁₂ are substituted by one or more substituents selected from halogen, hydroxy, C₁-C₄ alkyl, C₁-C₄ alkoxy, nitro, CN O—C(O)—C₁-C₄alkyl and C(O)—O—C₁-C₄-alkyl;
R₅, R₆ and R₇ are independently hydrogen, lower alkyl, aryl, aryl lower alkyl, cycloalkyl, or cycloalkyl lower alkyl, and
wherein the substituents on R₁, R₂, R₃, R₄, Q, and A and A₁ groups are independently halo, hydroxy, lower alkyl, lower alkenyl, lower alkynyl, lower alkanoyl, lower alkoxy, aryl, aryl lower alkyl, amino, amino lower alkyl, diloweralkylamino, lower alkanoyl, amino lower alkoxy, nitro, cyano, cyano lower alkyl, carboxy, lower carbalkoxy, lower alkanoyl, aryloyl, lower arylalkanoyl, carbamoyl, N-mono- or N,N-dilower alkyl carbamoyl, lower alkyl carbamic acid ester, amidino, guanidine, ureido, mercapto, sulfo, lower alkylthio, sulfoamino, sulfonamide, benzosulfonamide, sutfonate, sulfanyl lower alkyl, aryl sulfonamide, halogen substituted aryl sutfonate, lower alkylsulfinyl, arylsulfinyl; aryl-lower alkylsulfinyi, lower alkylarylsulfinyl, lower alkylsulfonyl, arylsulfonyl, aryl-lower alkylsulfonyl, lower aryl alkyl lower alkylarylsulfonyl, halogen-lower alkylmercapto, halogen-lower alkylsulfonyl, phosphono (—P(═O)(OH)₂), hydroxy-lower alkoxy phosphoryl or di-lower alkoxyphosphoryl, (R₉)NC(O)—NR₁₀R₁₃, lower alkyl carbamic acid ester or carbamates or —NR₈R₁₄, wherein R₈ and R₁₄ can be the same or different and are independently H or lower alkyl, or R₈ and R₁₄ together with the N atom form a 3- to 8-membered heterocyclic ring containing a nitrogen heteroring atoms and may optionally contain one or two additional heteroring atoms selected from nitrogen, oxygen and sulfur, which heterocyclic ring may be unsubstituted or substituted with lower alkyl, halo, lower alkenyl, lower alkynyl, hydroxy, lower alkoxy, nitro, amino, lower alkyl, amino, diloweralkyl amino, cyano, carboxy, lower carbalkoxy, formyl, lower alkanoyl, oxo, carbarmoyl, N-lower or N,N-dilower alkyl carbamoyl, mercapto, or lower alkylthio, and R₉, R₁₀, and R₁₃ are independently hydrogen, lower alkyl, halogen substituted lower alkyl, aryl, aryl lower alkyl, halogen substituted aryl, halogen substituted aryl lower alkyl.

18. A method according to claim 4, wherein where the IAP inhibitor compound is selected from N-1-Cyclohexyl-2-(2-[4-(4-fluoro-benzoyl)-thiazol-2-yl)-pyrrolidin-1-yl]-2-oxo-ethyl)-2-methylamino-propionamide; N-[Cyclohexyl-(ethyl-{1-[5-(4-fluoro-benzoyl)-pyridin-3-yl]-propyl}carbamoyl)-methyl]-2-methylamino-propionamide; N-(1-Cyclohexyl-2-{2-[5-(4-fluoro-phenoxy)-pyridin-3-yl]-pyrrolidin-1-yl)-2-oxo-ethyl)-2-methylamino-propionamide; and N-[1-Cyclohexyl-2-(2-(2-[(4-fluorophenyl)-methyl-amino]-pyridin-4-yl}pyrrolidin-1-yl]-2-oxo-ethyl)-2-methylamino-propinamide and pharmaceutically acceptable salt thereof.

19. A method according to claim 5, wherein where the IAP inhibitor compound is selected from N-1-Cyclohexyl-2-{2-[4-(4-fluoro-benzoyl)-thiazol-2-yl)-pyrrolidin-1-yl)-2-oxo-ethyl)-2-methylamino-propionamide; N-[Cyclohexyl-(ethyl-{1-[5-(4-fluoro-benzoyl)-pyridin-3-yl)-propyl]carbarmoyl)-methyl]-2-methylamino-propionamide; N-(1-Cyclohexyl-2-(2-[5-(4-fluoro-phenoxy)-pyridin-3-yl)-pyrrolidin-1-yl)-2-oxo-ethyl)-2-methylamino-propionamide; and N-[1-Cyclohexyl-2-(2-(2-[(4-fluorophenyl)-methyl-amino]-pyridin-4-yl}pyrrolidin-1-yl)-2-oxo-ethyl]-2-methylamino-propinamide and pharmaceutically acceptable salts thereof.
